(19) United States
(12) Patent Application Publication
(10) Pub. No.: US 2015/0141965 A1
(43) Pub. Date: May 21, 2015

(54) TUBING FOR MITIGATING AGAINST MICROBIAL MIGRATION AND METHOD AND SYSTEM FOR MAINTAINING CLOSED-SYSTEM OF URINARY TUBING

(71) Applicant: Celeste V. Bonham, Fredericksburg, TX (US)

(72) Inventor: Celeste V. Bonham, Fredericksburg, TX (US)

(21) Appl. No.: 14/281,524

(22) Filed: May 19, 2014

Related U.S. Application Data
(60) Provisional application No. 61/962,776, filed on Nov. 15, 2013.

(51) Int. Cl. A61M 25/00; A61M 39/24; A61M 39/16; A61M 39/10

(52) U.S. Cl. CPC A61M 25/0017; A61M 39/10; A61M 39/24; A61M 25/0009; A61M 39/162; A61M 2207/00; A61M 2202/0014; A61M 2202/0496
USPC 604/544; 29/428; 29/426.2

(57) ABSTRACT

A urinary tubing for mitigating against microbial migration, as well as a system and method for forming and maintaining a closed-system of urinary tubing are described. Such tubing may comprise: a first terminal end; a second terminal end, longitudinally opposing the first terminal end; a wettable region, which may comprise a surface which may be wetted by a fluid flowing within the tubing; and a means for mitigating against microbial migration in a direction opposite of intended flow. Such means may be selected from one or more of the group comprising at least one check-valve, at least one biofilm abater, and the wettable region treated with an antimicrobial coating. Such means may be located within the tubing, at the first terminal end, or at the second terminal end.

### PRIORITY NOTICE

The present application claims priority under 35 U.S.C. §119(e) to U.S. Provisional Patent Application Ser. No. 61/962,776 filed on Nov. 15, 2013, the disclosure of which is incorporated herein by reference in its entirety.

### STATEMENT REGARDING FEDERAL SPONSORSHIP

No part of this invention was a result of any federally sponsored research.

### TECHNICAL FIELD OF THE INVENTION

The present invention relates in general to urinary tubing and more specifically to flexible urinary tubing which may comprise various means for mitigating against microbial migration in a direction opposite of intended flow. Additionally, a system and a method for forming and maintaining a closed-system with respect to urinary tubing connected to a catheter are described and disclosed.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

One in five patients admitted to hospitals receive an indwelling urinary catheter (hereinafter, “catheter”). When a patient receives a catheter, and the longer the catheter is placed inside the body, the more likely the patient will develop a urinary tract infection (UTI). Hospital-associated infections (HAIs) are infections acquired during the course of receiving treatment for other conditions within a hospital setting. In the United States according to the Centers for Disease Control and Prevention (CDC) more than two million patients develop HAIs in the United States each year with about 99,000 of such cases resulting in death.

UTIs may be caused by microbes (e.g. bacteria) entering the body through the catheter. The distribution of microbes among patients with hospital-acquired urinary tract-related bloodstream infections may include: enterococcus, candida, E. coli, klebsiella, staphylococcus, and the like. According to the CDC, the urinary tract is the most common site of HAIs and accounts for more than 40% of the total number of HAI reported by acute-care hospitals. Catheter-associated urinary tract infection (CAUTI) is the most frequent HAI in the United States. In 2014, the CDC released a HAI Progress Report stating that among five categories of HAIs, only CAUTI had an increase of incidents. Due to the increase of CAUTIs, the Centers for Medicare and Medicaid Services (CMS) will not allow hospitals to charge patients for treating a hospital-acquired CAUTI because nosocomial CAUTIs and are believed to be “reasonably preventable.” The cost of treating UTIs could cost U.S. hospitals between $1.6 billion and $7.39 billion annually in lost Medicare reimbursements since treating each CAUTI incident may cost between US$600 to US$2,800 to treat.

Microbes may travel intraluminally in the urinary system at least two ways: (1) suspended and floating microbial cells within the urine (i.e. bacteriuria) where there is back-flow (reflux) of infected urine; and (2) by biofilm migration (colonies of microbial cells that form layers and attach themselves to surfaces) that can ascend up through urinary tubing surfaces and into the catheter and potentially enter the patient's body. Research currently suggests that preventing urine back-flow (reflux) from entering the catheter and/or incorporating a hurdle or barrier that prevents biofilms from ascending through the urinary collection system would help to mitigate the number of UTIs. Currently, the number of patients developing bacteriuria or UTIs after two and three days is 10% to 30%; while after one week or longer is near 90%, and; long-term catheterization (of one month or more) results in near 100% of patients with bacteriuria or UTIs. Although not all CAUTIs may be prevented, it is believed by the medical community that a large number of CAUTIs may be avoided by the proper management of indwelling and other catheters. CMS developed a list of recommendations and guidelines to help reduce UTIs and CAUTIs. One of these recommendations included maintaining a “closed-system” for indwelling catheters and means to prevent back flow of urine which could contain bacteriuria or other microbes. Furthermore, prevention may be the best way to manage nosocomial UTI, as opposed to focusing on expensive treatment, which may or may not be effective, as many microbes are becoming increasingly less sensitive and more resistant to antibiotics.

A “urinary system” or more simply a “system” as used in this specification, unless otherwise stated, may comprise: a patient (including their urethra, bladder, and/or kidneys), a catheter connected to the patient, and a length of urinary tubing connected to the catheter, and sometimes a urine bag may also be defined as part of the system. A “closed-system” as used in this specification, unless otherwise stated, may refer to the concept that the system as defined above may be formed and maintained isolated, i.e. kept physically separated, from components and microbes outside of the system. Thus a closed-system may be beneficial because by being closed, microbes are denied various routes of entry into the patient. If such a closed-system is breached (e.g. by inappropriate opening), then microbes may enter the tubing and catheter and travel up into the urethra or body wall of the patient and infect the patient.

To date (circa 2014), the inventor is not aware of any prior art that specifically addresses a device or component that forms a closed-system, or where such a device or component may prevent urine back-flow (reflux) of urine located within urinary tubing that connects the urinary indwelling catheter to the urine bag, or where such a device or component may prevent biofilm migration. Reference should also be made that there are no commercial products currently available that specifically addresses maintaining a closed-system by placing a check-valve inside the urinary tubing that connects the urinary catheter to the urine bag.

Rather, unrelated prior art consists of inventions for closed-system irrigation connectors, intravenous syringe ports, closed adapters for enteral formula delivery, and needleless IV access ports for small bore luers—i.e. none of this prior art deals with urinary systems.

For example, some such prior art include: maintaining a closed-system for blood and urine specimens (i.e. for samples already taken, which has nothing to do with maintaining a urinary system as closed) and maintaining a closed-system for irrigation; and of using check-valves in feeding tubes. Such prior art does not incorporate any check-valve within the primary urinary tubing running from the indwelling catheter to the urine bag. Another prior art example includes a device that disengages the indwelling catheter to avoid the indwelling catheter from being pulled from the wall of the patient.

Additionally, the prior art may include a check-valve located within a urine bag. This prior art may prevent urine back flow from the urine bag into the tubing, but only when the urine bag is connected to the tubing. However, if the tubing and the urine bag are disconnected there is no barrier for prevention of bacteria entering the tubing and catheter and the closed-system is breached. In fact, when a urine bag is detached from a terminal end of urinary tubing, what may have been a closed-system with respect to the urinary system may now be breached. Accidents occur in hospital settings and may occur while empting urine or taking a urine sample from a urine bag. If there is no anti-reflux valve (i.e. check-valve) to prevent such incidents, resulting in compromised closed-systems, then microbes in the urine and airborne, may contaminate the urine collection/drainage system and cause vulnerability to the patient for a CAUTI.

There is a need in the art for satisfactorily addressing and reducing the high percentage of UTIs and CAUTIs that occur with current indwelling urinary catheter use.

It is to these ends that the present invention has been developed.

### BRIEF SUMMARY OF THE INVENTION

To minimize the limitations in the prior art, and to minimize other limitations that will be apparent upon reading and understanding the present specification, the present invention describes a tubing for mitigating against microbial migration, as well as a system and method for forming and maintaining a closed-system of urinary tubing.

Such tubing may comprise: a first terminal end; a second terminal end, longitudinally opposing the first terminal end; a wettable region, which may comprise a surface which may be wetted by a fluid flowing within the tubing; and a means for mitigating against microbial migration in a direction opposite of intended flow. Such means may be selected from one or more of the group comprising at least one check-valve, at least one biofilm abater, and the wettable region treated with an antimicrobial coating. Such means may be located within the tubing, at the first terminal end, or at the second terminal end.

It is an objective of the present invention to provide urinary tubing that may be capable of mitigating against microbial migration in a direction opposite of intended flow, by utilizing the various means discussed herein, specifically in the DETAILED DESCRIPTION OF THE INVENTION section of this specification.

It is another objective of the present invention to provide urinary tubing that may be capable of mitigating against urine back-flow (reflux) from the intended flow of urine within the urinary tubing and preventing back-flow into a catheter.

It is another objective of the present invention to provide urinary tubing that may comprise at least one check-valve positioned within the urinary tubing, at the first terminal end, and/or the second terminal end to mitigate against urine back-flow (reflux) from the intended flow of urine within both the urinary tubing and within the catheter.

It is another objective of the present invention to provide urinary tubing that may comprise at least one biofilm abater positioned within the urinary tubing to mitigate against biofilm migration across the at least one biofilm abater. See the DETAILED DESCRIPTION OF THE INVENTION section for a definition of a biofilm abater.

It is another objective of the present invention to provide urinary tubing that may comprise at least one wettable region (e.g. a region of urinary tubing inside diameter) treated with an antimicrobial coating positioned within the urinary tubing to mitigate against biofilm migration across the at least one wettable region treated with the antimicrobial coating.

It is another objective of the present invention to provide urinary tubing that may comprise various connectors, both with and without check-valves, where such connectors may be positioned within the urinary tubing, at the first terminal end, and/or the second terminal end, wherein such connectors (and/or tubing) may be treated (e.g. by coating) with an antimicrobial coating to mitigate against biofilm migration across such connectors treated with the antimicrobial coating.

It is another objective of the present invention to provide a method or series of methods for forming and maintaining a closed-system with respect to urinary tubing connected to a catheter.

It is yet another objective of the present invention to provide exemplary systems for forming and maintaining a closed-system with respect to urinary tubing connected to a catheter.

These and other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Elements that are known to be common and well understood to those in the industry are not depicted in order to provide a clear view of the various embodiments of the invention.

FIG. 1(a) depicts an exemplary embodiment of urinary tubing (hereinafter, “tubing”) connected at one end to a catheter and at the other end to a urine leg bag, shown from a frontal view. The catheter is shown as connected to a patient.

FIG. 1(b) depicts an exemplary embodiment of tubing connected at one end to a catheter and at the other end to a urine bed bag, shown from a longitudinal side view. Here the catheter is connected to a patient that is lying down on a bed.

FIG. 1(c) depicts an exemplary embodiment of tubing connected at one end to a catheter and at the other end to a urine bag, shown from a top view.

FIG. 2 depicts an exemplary embodiment of tubing comprising a connector integrated with check-valve, where one end of the tubing is connected to an exit port of a catheter via the connector integrated with check-valve, shown from a top view.

FIG. 2(a) depicts the exemplary embodiment of the tubing of FIG. 2, shown from a close up longitudinal cross-sectional view.

FIG. 2(b) depicts an exemplary embodiment of the tubing of FIG. 2(a), but with an addition of tape wrapping a connection between the catheter and the tubing, shown from a longitudinal cross-sectional view.

FIG. 3(a) depicts an exemplary embodiment of tubing which may comprise a connector with a non-integral check-valve, where one end of the tubing is connected to an exit port of a catheter via the connector with the non-integral check-valve, shown from a longitudinal cross-sectional view.

FIG. 3(b) depicts an exemplary embodiment of the tubing of FIG. 3(a), but with an addition of tape wrapping a connection between the catheter and the tubing, shown from a longitudinal cross-sectional view.

FIG. 4 depicts an exemplary embodiment of tubing, but where the tubing may be subdivided at a joint into two smaller tubes such that a connector integrated with check-valve may be inserted between the two smaller tubes to join the two smaller tubes, which then form the tubing, shown from a top view.

FIG. 4(a) depicts the exemplary embodiment of tubing of FIG. 4, shown from a longitudinal cross-sectional view.

FIG. 4(b) depicts an exemplary embodiment of the urinary tubing of FIG. 4(a), but where the joint is circumscribed by a coupling sleeve, shown from a longitudinal cross-sectional view.

FIG. 5(a) depicts an exemplary embodiment of tubing, but where the tubing may be sub-divided at a joint into two smaller tubes such that a connector with non-integral check-valve may be inserted between the two smaller tubes to join the two smaller tubes, which then form the tubing, shown from a longitudinal cross-sectional view.

FIG. 5(b) depicts an exemplary embodiment of the urinary tubing of FIG. 5(a), but where the joint is circumscribed by a coupling sleeve, shown from a longitudinal cross-sectional view.

FIG. 6(a) depicts an exemplary embodiment of tubing where a check-valve may have been inserted into the tubing by pushing the check-valve into a desired location, shown from a longitudinal cross-sectional view.

FIG. 6(b) depicts an exemplary embodiment of tubing with a biofilm abater inserted into the tubing, shown from a longitudinal cross-sectional view.

FIG. 6(c) depicts an exemplary embodiment of tubing with a biofilm abater inserted into the tubing, shown from a top cross-sectional view.

FIG. 6(d) depicts an exemplary embodiment of tubing with a check-valve and with a biofilm abater inserted into the tubing (upstream of) the check-valve, shown from a longitudinal cross-sectional view.

FIG. 6(e) depicts an exemplary embodiment of tubing where an inner region of the tubing has an antimicrobial coating, and where the coated inner region is upstream of a check-valve, shown from a longitudinal cross-sectional view.

FIG. 6(f) depicts an exemplary embodiment of tubing where there are two check-valves inserted into the tubing, in serial fashion, shown from a longitudinal cross-sectional view.

FIG. 6(g) depicts an exemplary embodiment of tubing which may comprise a sampling port, shown from a longitudinal cross-sectional view.

FIG. 6(h) depicts an exemplary embodiment of tubing which may comprise a graphical indicator which may indicate which end of the tubing may be detached from a urine bag, shown from a longitudinal cross-sectional view.

Note, with respect to the above stated cross-sectional views, cross-sections of the various connectors with integral check-valves, connectors with non-integral check-valves, and check-valves without connectors are not depicted. Cross-section of the various catheter sampling ports are also not depicted.

Note, any breaks depicted in tubing length in the various figures may indicate that the tubing may have a variety of lengths.

### DETAILED DESCRIPTION OF THE INVENTION

Various exemplary embodiments of a tubing configured to mitigate against microbial migration in a direction opposite of intended flow are described and disclosed. A system and a method for forming and maintaining a closed-system with respect to urinary tubing connected to a catheter are also described and disclosed.

Before turning to a discussion of the invention's various exemplary structures, disclosures regarding various definitions and functional objectives of the inventive structures are disclosed. “Intended flow,” “microbe, “microbial,” “antimicrobial,” “system,” “closed-system,” and “tubing” are all defined below and in turn.

An example of “intended flow” may be a flow of urine beginning from a patient's urethra (or bladder or kidney) and flowing into and through a urinary catheter (through an inside diameter), then into urinary tubing (through an inside diameter), and finally into a urine bag (e.g. leg or bed). Such intended flow is generally accomplished by the catheter being placed within the patient's urethra (or bladder or kidney), followed by the urinary tubing connected to an exit port of the catheter, and then with the urine bag connected at a remaining terminal end of the urinary tubing, and where the intended flow may be accomplished by each subsequent component being placed below the immediately prior component, i.e. to facilitate flow downhill. Any urine flow opposite of this intended flow direction is known as back-flow or reflux. And a primary objective of this invention is to mitigate against microbes migrating in the direction opposite of the intended flow. For example, one way to mitigate against undesirable microbe migration, may be to prevent or mitigate against fluid (e.g. urine) back-flow (reflux), since such fluid may be carrying microbes.

However, regardless of fluid flow, microbes may migrate against the direction of intended flow by forming a biofilm on a surface of the inside of tubing and then said biofilm growing (migrating) in the direction opposite of intended flow. Such surface growth of microbes may generally be facilitated by the microbes attaching to a wettable surface and then growing on the wettable surface. Thus, a second way to mitigate against undesirable microbe migration may be to inhibit the growth of microbes on such surfaces of the tubing, especially wettable surfaces of the tubing. Additionally, a third way to mitigate against undesirable microbe migration may be to inhibit attachment of microbes on such surfaces of the tubing.

Note, in this specification, “microbes” and “microbial” refers to a variety of micro-organisms which may comprise: bacteria, fungi (e.g. yeast), viruses, protozoans, and the like. Microbes may be floating freely and/or suspended within a fluid, including fluid flowing within tubing. Microbes attached to a surface may form a colony comprising many individual microbial cells, i.e. a biofilm. The term, “biofilm” refers to at least one microbial cell that has attached itself to a surface, i.e. a biofilm may be a microbial colony of many microbial cells attached to a surface.

The term, “antimicrobial” as used in this specification may refer to mitigating against microbe migration in a direction opposite of intended flow. Functionally, this invention may provide for at least three mechanisms to this desired objective of providing an antimicrobial tubing and system, where those three mechanisms may be: (1) mitigating against fluid back-flow (reflux); (2) mitigating against microbe growth; and (3) mitigating against microbe attachment to surfaces, particularly to wettable surfaces. As used in this specification, the term “mitigate” may be synonymous with “abate” and may include and encompasses the terms “prevent” and “inhibit.”

In various exemplary embodiments, a “system” as used in this specification may comprise: a catheter and a length of urinary tubing. In other exemplary embodiment, a “system” as used in this specification may comprise: a catheter, a length of urinary tubing, and a urine bag. In exemplary embodiments, a “system” as used in this specification may comprise: a patient, a catheter, and a length of urinary tubing. In exemplary embodiments, a “system” as used in this specification may comprise: a patient, a catheter, a length of urinary tubing, and a urine bag. In all the above definitions of system, the urinary tubing may further comprise some additional structures (e.g. check-valves), some with various antimicrobial structures and functions, as further disclosed and discussed below.

This invention may comprise the various exemplary embodiments of such a tubing (e.g. urinary tubing as disclosed herein). This invention may also comprise the various systems, wherein such systems may comprise the tubing and the catheter. In other words, the tubing as the invention may not comprise the catheter; but, the system as the invention may comprise the catheter.

With respect to the catheter, such catheters may comprise both indwelling and external catheters. Indwelling catheters may comprise Foley (i.e. inserted into the urethra), suprapubic (inserted into abdomen or bladder), and nephrostomy. External catheters may comprise condom catheters and female external collection systems.

With respect to the catheter, a connection between the catheter and the tubing may be located at the catheter's “exit port.” Such an exit port may also be referred to as a “drainage port.”

A “closed-system” as used in this specification may refer to how the closed-system may be formed and maintained isolated, i.e. kept physically separated, from components outside of the system. It may be desirable to maintain such systems as closed to help prevent infection, including urine back flow, from microbes entering the system and making their way into a patient.

A closed-system may be formed by maintaining the following connected components, with at least three points of connections (or points of being sealed from outside influence): (1) a catheter properly connected to a patient's urethra (or bladder or kidney); (2) a proper connection between the catheter's exit port and a terminal end of urinary tubing (e.g. a first terminal end); and (3) the remaining terminal end (e.g. second terminal end) of the urinary tubing being connected to a urine bag, or otherwise being sealed (e.g. taped, capped off or clamped shut). Each of these three points of connection may be a source of breach to render an otherwise closed-system into an open system. For example, if the catheter becomes disengaged from the patient's urethra, the system may then be open and microbes may then enter into the catheter and the human body through the urethra. Another example, if the catheter exit port becomes disengaged from a terminal end of tubing (e.g. a first terminal end), then microbes may enter into what may now be an open system. This connection should be taped to avoid unintended disconnect. With respect to the remaining terminal end of tubing (e.g. second terminal end), when such a terminal end is properly connected to a urine bag, and the other two connections are in place, then the system may be closed and no urine backflow can enter the tubing due to the check valve (anti-reflux) in the urine bag. But unless some additional step or mechanism is utilized when the urine bag is disengaged from the system, for example, to be changed, the system may be open and microbes may then enter the system because the remaining terminal end of tubing (e.g. second terminal end) is now open to the external environment since the means of prevention of urine back flow is located with the urine bag that has been removed. This invention, in its various exemplary embodiments, may provide structure to maintain the system as closed even when the tubing is disconnected from the urine bag.

“Tubing” as used in this specification may have several different meanings. Functionally, tubing may be a conduit for transporting a material from one point to another point. For example, that material may be a fluid, such as urine. Structurally, tubing in a traditional sense may comprise an elongated hollow member (e.g. a cylinder) with at least one length (with at least two terminal ends), an outside diameter, an inside diameter, and a wall thickness (defined by the difference in outside and inside diameters). The length of tubing may generally be linear, but could form other shapes, such as a “Y” shape.

Tubing in this invention may further comprise additional structure such as: various connectors, both with and without check-valves; various check-valves, both with and without connectors; biofilm abaters; and antimicrobials coatings: of tubing surfaces, of connectors, and of check-valves. Tubing as used in this specification may be flexible tubing, i.e. as opposed to rigid tubing. Tubing as used in this specification may be medical grade tubing. Medical tubing may be sub-divided into urinary tubing, for the transport of urine from a catheter to a urine bag. Tubing as used herein may be described as flexible urinary tubing. Tubing may run from an exit port of the catheter directly to the urine bag, i.e. a primary length of tubing. Tubing as used herein may be described as flexible indwelling catheter tubing. The length of tubing may vary to accommodate a leg bag or longer to accommodate the distance between the catheter (or patient) and the bed bag.

In various exemplary embodiments, tubing may be substantially constructed of various polymers. The polymers may be suitable for tubing extrusion, injection molding, ultrasonic bonding, solvent bonding, heat welding, and/or chemical adhesives. For example, tubing in the traditional sense of an elongated hollow member with an outside and inside diameter may be efficiently manufactured by extrusion into various lengths. Whereas, tubing in the traditional sense may also be injection molded, but where such a means of manufacture may be more expensive and with limited available lengths compared to extrusion methods of manufacture. Additional, structural components of the tubing (e.g. connectors with or without check-valves, check-valves with or without connectors, biofilm abaters, and coupling sleeves) may be substantially constructed using injection molding.

Such polymers may comprise: urethane (including polyurethanes), rubber (with or without latex), polyvinyl chloride (PVC), silicone, polyethylene (low density and high density), nylon, fluropolymers, polypropylene, acrylonitrile-butadiene styrene (ABS), polycarbonate, acrylic, and the like. PVC may be the most common material of construction for flexible medical urinary tubing.

With respect to use of “substantially constructed of” in the above materials discussion, such phrasing may be used because various exemplary embodiments of tubing may also include some additional non-polymer materials. For example, in various exemplary embodiments there may be antimicrobial coatings to certain regions of the tubing, to connectors, to check-valves and/or to biofilm abaters. There may some metal (e.g. stainless steel) used in some components, for example, some check-valves may employ springs made of metal. Biofilm abaters may be constructed of entirely of silver (or silver alloy) or coated with silver (or silver alloy). In some embodiments, adhesives tapes (as a type of coupling sleeve) may also be a component of the tubing. Some solvents may be used for solvent bonding and chemical adhesives may also be utilized in assembly.

Polymer formulations may also comprise other ingredients which increase the cured polymers antimicrobial properties, for example and without limiting the scope of the present invention, including silver within the polymer formulation.

More than one type of polymer may be used within a given exemplary embodiment of tubing. For example, and without limiting the scope of the present invention, in various exemplary embodiments, the elongated member of the tubing may be substantially constructed of PVC, while a connector, with or without check-valve, may be substantially constructed of HDPE (high density polyethylene) or polycarbonate.

With respect to materials of construction because the tubing may be medical grade tubing, the choice of materials may be limited to polymers which may be manufactured aseptically (e.g. in clean rooms) and then subsequently sterilized without the tubing significantly degrading. Common sterilization methods include steam sterilization via autoclaves, gamma irradiation, ultraviolet exposure, and ethylene oxide (EtO) gas exposure. Gamma irradiation tends to render materials more brittle and steam sterilization may leave behind water vapor which condenses and may facilitate microbial contamination subsequent to the sterilization. Each of the above listed polymers may have various formulations that when cured may appropriately be sterilized by each of these sterilization methods.

Choice of materials may also be limited to polymers which may be field sterilized or sanitized by exposure to various chemicals, such as alcohol (e.g. isopropyl alcohol), bleach, and peroxides. (Field sterilization or sanitization may be sterilization or sanitization done by the user of the product, such as a medical practitioner, as opposed to sterilization that occurs as a step in the manufacturing process.)

Note, with respect to the materials of construction, it is not desired nor intended to thereby unnecessarily limit the present invention by reason of such restricted disclosure.

Now turning to a general discussion of tubing structure, which is further detailed in the discussion of the various figures. In various exemplary embodiments, the tubing may comprise a first terminal end and a second terminal end, such that the first terminal end may be disposed opposite of the second terminal end, e.g. located at longitudinal opposing ends. Tubing may be bounded by the first terminal end and the second terminal end.

In some embodiments, the tubing may comprise a wettable region, which may comprise a surface which is wetted when a fluid flows within the tubing. For example, and without limiting the scope of the present invention, common wettable regions may comprise the interior surfaces of the tubing which may comprise an inside diameter of the tubing along a corresponding length of the tubing. Additional wettable regions of the tubing may comprise various interior surfaces of various connectors, with and without check-valves, as well as check-valves without connectors.

An outside diameter of the tubing is generally not a wettable region nor a wettable surface. However, the outside diameter regions immediately proximal of the first terminal end and the second terminal end may be physically so close to wettable regions of the tubing, that such outside diameter regions may also be ideal candidate regions for treating with an antimicrobial coating.

In some embodiments, the tubing may comprise a means for mitigating against microbial migration in a direction opposite of intended flow. The means for mitigating against microbial migration in the direction opposite of intended flow may be selected from one or more of the group comprising: (1) at least one check-valve; (2) at least one biofilm abater; (3) and the wettable region treated with an antimicrobial coating. For example, and without limiting the scope of the present invention, the tubing may comprise one check-valve which has been treated with an antimicrobial coating (or one check-valve with no antimicrobial coating). Any combination of these three means may be located within the tubing and/or at one or both of the terminal ends (first terminal end and second terminal end). Each of these three antimicrobial means for mitigating against microbial migration in a direction opposite of intended flow is briefly discussed below.

Check-valves as used in this specification refer to devices which may be intended to allow fluid flow in only one direction. Check-valves may accomplish this function using a variety of means well known in the art, such as utilizing springs with balls, flaps (diaphragms), one-way gates (swing and/or tilt), duckbills, and the like. A given check-valve may generally have at least one inlet and at least one outlet, where the inlet and the outlet are generally points of connection to the check-valve. Check-valves may be in an open configuration when no back pressure is applied to the check-valve, permitting flow in the desired direction. Check-valve locations in some embodiments may be positioned at either terminal end of the tubing, both terminal ends of the tubing, or in between the two terminal ends of the tubing.

For example, a check-valve used in urinary tubing, as described and disclosed in this specification, permits urine flow in the desired direction when the upstream urine pressure exceeds the check-valve's resting state and opens the check-valve. Upstream urine pressure may be created naturally from the patient urinating, or may arise by virtue of a static head, i.e. the height of urine in tubing (and catheter) upstream of a check-valve, where the greater the height of urine, the greater the urine pressure (greater the static head of urine pressure). Such upstream urine pressure created by a static head of urine is with respect to a gravitational pull, i.e. urine like all liquid fluids flows downhill. If the exit end of urinary tubing is raised above the entry point of urinary tubing, any urine within the urinary tubing may be encouraged to flow backwards, against the intended flow, because by raising the exit end above the entry end greater downstream urine pressure has been created by a static head of urine. A check-valve in proper place (e.g. not installed backwards) within the tubing may prevent such back-flow (reflux) because the check-valve may be designed to be closed when the downstream pressure exceeds the upstream pressure.

Note, the locational identifiers of “downstream” and “upstream” may be in reference to the direction of intended flow, i.e. intended flow flows from the upstream to the downstream. Such locational identifies may also be in reference to some third point in between the upstream and downstream locations, such as a check-valve.

Now turning to the second antimicrobial means for mitigating against microbial migration in a direction opposite of intended flow, the use of a biofilm abater within the tubing. As used in this specification, the biofilm abater is a device which may prevent or mitigate against biofilm movement (migration or growth) in the direction opposite of intended flow. Because biofilm movement (e.g. migration or growth) occurs more readily on wettable regions within the tubing, particularly regions which are currently wet, then the biofilm abater may be located within the tubing so as to prevent or mitigate against biofilm movement in the direction opposite of intended flow.

In some embodiments, biofilm abaters may circumscribe an outside diameter of the tubing.

Biofilm abaters may operate in one of two ways, which are not mutually exclusive, i.e. either or both methods may be simultaneously employed. First, the biofilm abater may inhibit microbe growth. Secondly, the biofilm abater may inhibit microbe attachment to a surface (generally a wettable surface). Inhibiting growth may involve interfering with a microbe's cellular processes, such as cellular replication or cell-wall development. While inhibiting attachment may involve creating a surface substrate that is molecularly too slippery for a microbe to attach to.

In either mechanism, growth inhibiting or attachment inhibiting, the biofilm abater may take on the structural and geometric properties of a ring which is configured to fit snuggly within the tubing. The biofilm abater may comprise a ring. The ring may comprise an outside diameter that may be in direct physical contact with the tubing's inside diameter, such that the tubing's inside diameter frictionally grips the ring's outside diameter. Such a ring structure may also comprise an inside diameter, configured to permit fluid flow. This point of direct physical contact may also be such that no fluid is permitted to flow between the ring's outside diameter and the tubing's inside diameter. All fluid flow may be directed through the inside diameter of the ring.

Such a ring may inhibit biofilm growth on the wettable surfaces of the ring, such as the ring's inside diameter, by the ring's wettable surfaces comprising an antimicrobial property that inhibits growth. For example, in various exemplary embodiments, the ring may be made entirely of silver (or a silver alloy) or the ring may be coated with silver (or silver alloy). Silver, in both metallic form (and alloys) and silver salt forms, is well known within the art of comprising antimicrobial properties which inhibit microbial growth and may actually kill microbes. The biofilm abater which may comprise the ring, wherein the ring may comprise silver or a silver coating may inhibit biofilm migration across the inside diameter of such a ring. Such a ring may be entirely constructed of metallic silver or various external surfaces of the ring may be coated with silver. Such antimicrobial properties are not limited to silver and silver coatings.

Such a ring structure may inhibit biofilm attachment to the wettable surfaces of the ring, such as the ring's inside diameter, by the ring's wettable surfaces comprising an antimicrobial property that inhibits attachment. In various exemplary embodiment, the ring's wettable surfaces, such as the ring's inside diameter may be coated with a material providing anti-attachment properties. Anti-attachment may be accomplished by forming a surface that is molecularly smooth (molecularly slippery), such that there is no molecular geometry for microbes to attach to. For example, surfaces may be treated with a Teflon® coating, which is known to result in a slippery surface that reduces microbe attachment. Other chemicals may also be used to treat surfaces yielding a molecularly smooth surface that microbes find difficult to attach to. Such coating treatments may be applied to materials of construction typical for tubing, various connectors, and check-valves, such as silicone, PVC (polyvinylchloride), PU (polyurethane), and the like.

In various exemplary embodiments, a biofilm abater may comprise a ring, which may comprise a molecularly smooth coating of the ring's wettable surfaces, which may then inhibit biofilm migration across the inside diameter of such a ring because the microbes find difficulty in attaching to such coated regions. In various exemplary embodiments, the ring's outside diameter may not be treated with the molecularly smooth coating, as such a coating may interfere with the tubing's inside diameter frictionally gripping the ring's outside diameter.

Now turning to the third antimicrobial means for mitigating against microbial migration in a direction opposite of intended flow, which may be regions of the tubing which may comprise an antimicrobial coating, where such regions are predominantly wettable regions. Such coated regions of tubing may function in an equivalent manner as the biofilm abater embodiments discussed above, but instead of coating a separate component like the biofilm abater's ring, here a region or all of the tubing material of construction may be coated with one of the antimicrobial coatings. As noted above the antimicrobial coatings may either inhibit biofilm growth across the treated region or the antimicrobial coating may inhibit attachment to the coated region. Or the antimicrobial treated region may comprise both functions. For example, in various exemplary embodiments, the coated region may comprise a silver coating which may inhibit biofilm growth across the coated region. Whereas, in other exemplary embodiments, the coated region may comprise a molecularly smooth coating which may inhibit biofilm attachment to the coated region and thus inhibit biofilm migration across the coated region.

Coated regions may comprise a given length of the tubing's inside diameter, such that the coated region completely circumscribes the tubing's inside diameter for that given length, thus presenting a uniform barrier to biofilm migration (movement). Wettable treated regions may also comprise various interior surfaces of various connectors, with and without check-valves, as well as check-valves without connectors. For example, and without limiting the scope of the present invention, in various exemplary embodiments a check-valve's interior wettable surfaces may be coated with the antimicrobial coating.

In various exemplary embodiments, a coated region may comprise a region of outside diameter tubing that is immediately proximal of the first terminal end and/or of the second terminal end, as such outside diameter regions although are generally non-wettable regions, they are regions which are nevertheless prone to contaminating urinary tubing due to their close proximity and access to inside diameter wettable regions of the tubing. Such a distance of “immediately proximal” may be up to and including 8 inches from either terminal end (first terminal and/or second terminal end). In some embodiments, an entirety of the tubing may be coated with the antimicrobial material.

In some embodiments, the tubing material of construction itself, i.e. not just the surface regions, may have antimicrobial ingredients added into the materials formulation to yield a cured material that exhibits antimicrobial properties.

Each of these three means for mitigating against microbial migration in the direction opposite of intended flow may be located within the tubing at various locations along the tubing's length, at the tubing's first terminal end, or at the tubing's second terminal end.

In the following discussion that addresses a number of embodiments and applications of the present invention, reference is made to the accompanying figures (drawings) that form a part thereof, where depictions are made, by way of illustration, of specific embodiments in which the invention may be practiced. It is to be understood that other embodiments may be utilized and changes may be made without departing from the scope of the invention.

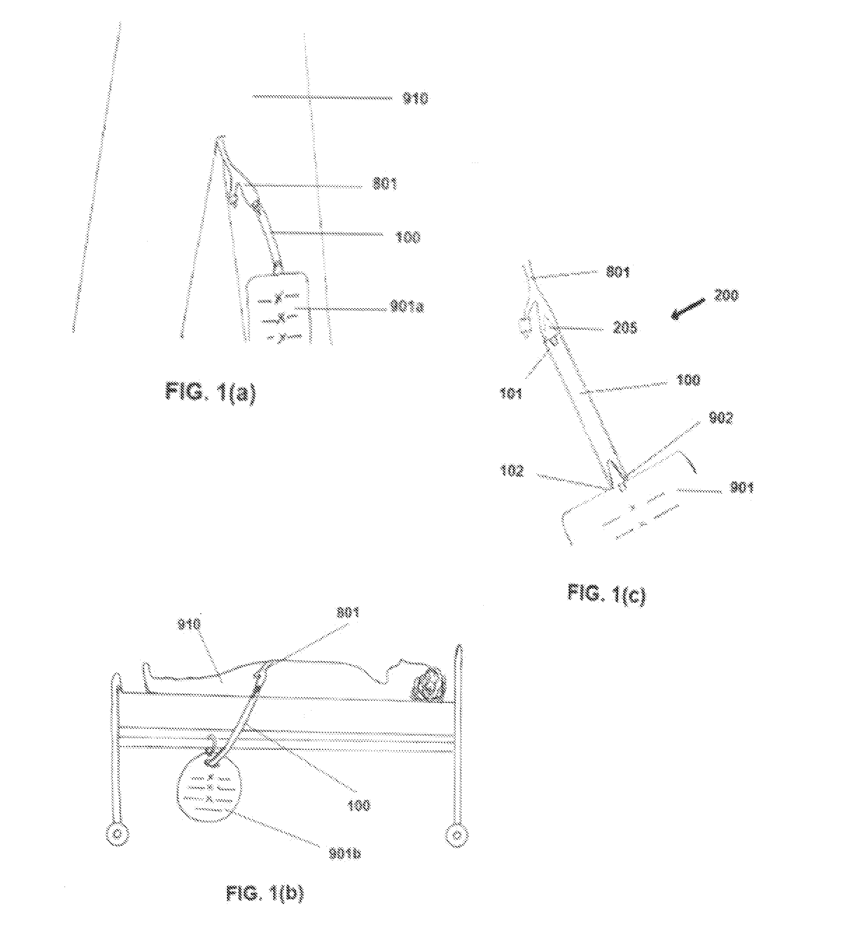

The FIG. 1 series of figures includes FIG. 1(a), FIG. 1(b), and FIG. 1(c). The FIG. 1 series of figures may serve two functions: (1) to place the tubing invention into context of being connected to a catheter at one end and a urine bag at an opposing end; and (2) to depict the inventive system, wherein the system may comprise the inventive tubing, as well as a catheter.

FIG. 1(a) depicts a tubing 100 connected at one end to a catheter 801 and at the other end to a urine leg bag 901a, shown from a frontal view. Catheter 801 as shown may be properly connected to patient 910, i.e. by proper partial insertion into patient 910's urethra, i.e. catheter 801 may be an indwelling catheter, such as a Foley catheter. FIG. 1(b) depicts tubing 100 connected at one end to catheter 801 and at the other end to a urine bed bag 901b, shown from a longitudinal side view. Here catheter 801 may be properly connected to patient 910 that is lying down or otherwise occupying a bed. FIG. 1(c) depicts an exemplary embodiment 200 of tubing 100 connected at one terminal end, a first terminal end 101 to catheter 801, and at the other terminal end, a second terminal end 102 to urine bag 901, shown from a top view.

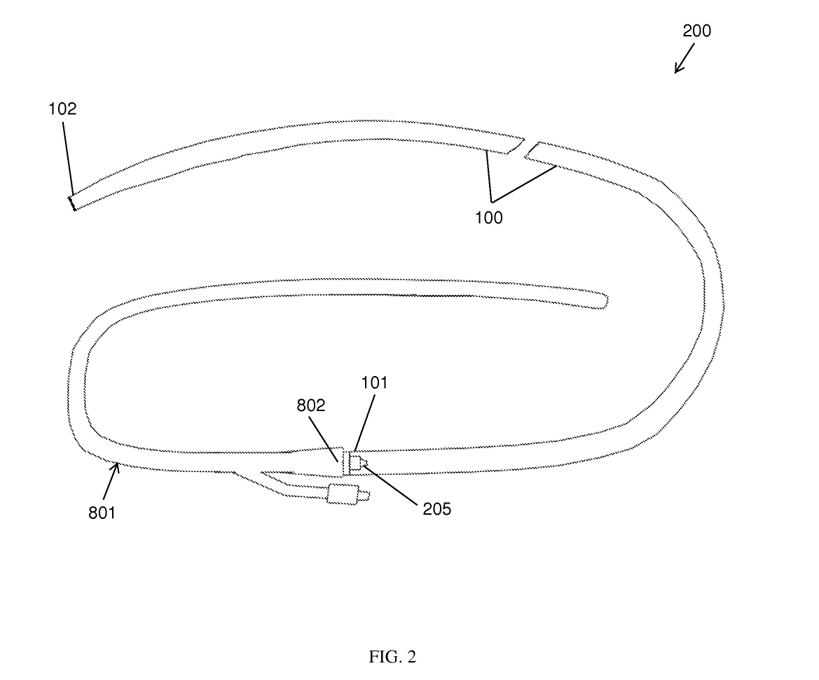

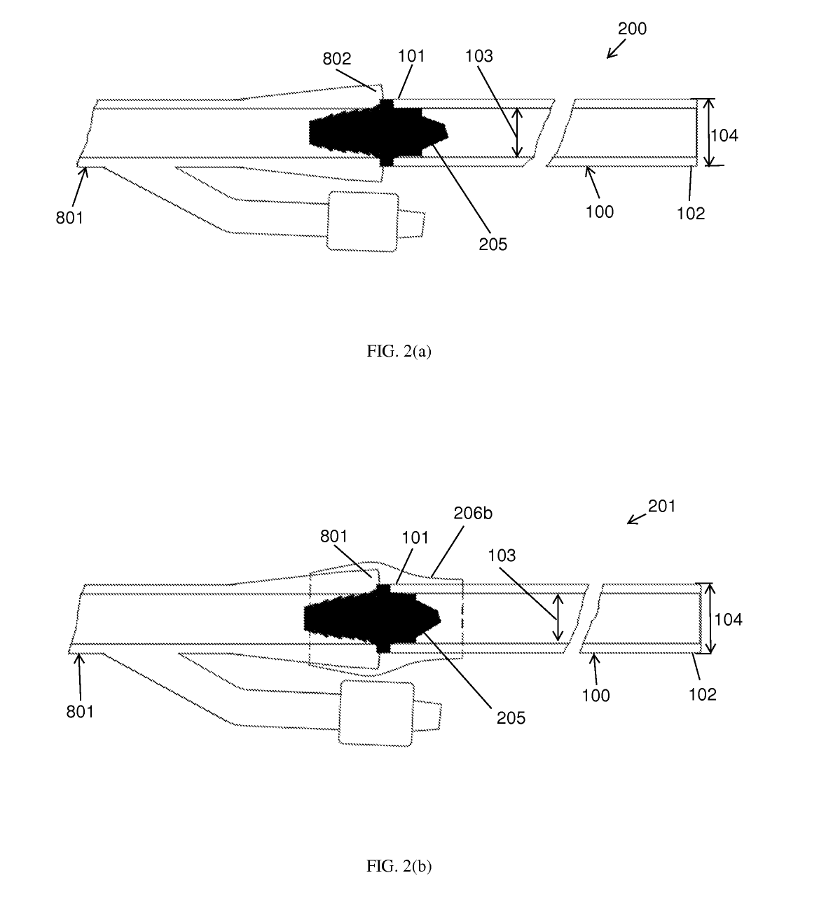

FIG. 2 depicts exemplary embodiment 200 of tubing 100 comprising a connector integrated with check-valve 205, where first terminal end 101 of tubing 100 may be connected to an exit port 802 of catheter 801 via connector integrated with check-valve 205, shown from a top view.

Note in FIG. 2 the entire catheter 801 and the entire tubing 100 may be depicted; while patient 910 and urine bag 901 are not depicted. Whereas, in FIG. 2(a), and FIG. 2(b) the focus may be depicting the connection where tubing 100 may be connected to catheter 801, which may utilize connector integrated with check-valve 205 to connect exit port 802 to first terminal end 101 of tubing 100.

In terms of tubing 100 overall length, such an overall length may be short to accommodate use with leg bag 901a. For example, and without limiting the scope of the present invention, such an overall length of tubing 100 may be from two inches to seven inches. In other embodiments, overall length may be twenty-four inches or longer to accommodate use with bed bag 901b.

FIG. 2(a) depicts exemplary embodiment 200 of tubing 100, shown from a close up longitudinal cross-sectional view of the connection. FIG. 2(b) depicts exemplary embodiment 201 of tubing 100 with an addition of tape 206b wrapping around the connection between catheter 801 and tubing 100, shown from a longitudinal cross-sectional view.

First general structure which may be common to both exemplary embodiment 200 and 201 is discussed, followed by additional structure of exemplary embodiment 201. Exemplary embodiment 200 of tubing 100 may comprise: first terminal end 101, second terminal end 102, and connector integrated with check-valve 205. First terminal end 101 may longitudinally oppose second terminal 102. Connector integrated with check-valve 205 may be a single article of manufacture. Connector integrated with check-valve 205 may be located at first terminal end 101 and may be configured to connect tubing 100 to catheter 801. The connection between catheter 801 and tubing 100 may be formed by connecting first terminal end 101 to exit port 802 via connector integrated with check-valve 205. Connector integrated with check-valve 205 may comprise two opposing ends, where one end may be sized to frictionally and removably couple with an inside diameter of first terminal end 101 and where the other end may likewise be sized to frictionally and removably couple with an inside diameter of exit port 802. And the check-valve component of connector integrated with check-valve 205 may be located internally within connector integrated with check-valve 205, and may prevent urine back-flow (reflux), which then serves as one example of a means for mitigating against microbial migration in a direction opposite of intended flow.

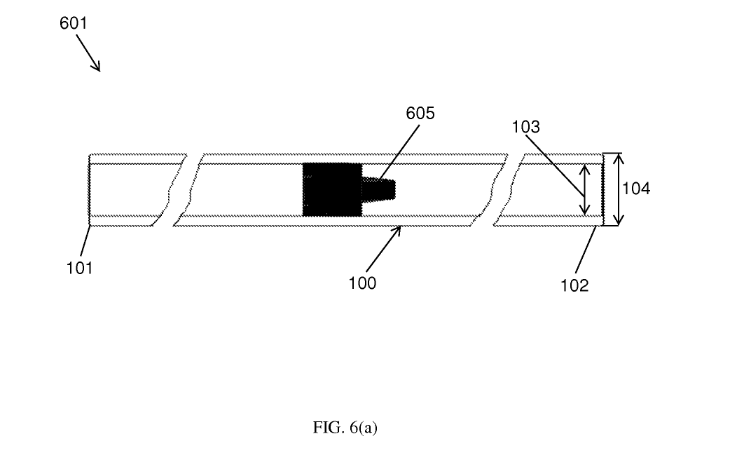

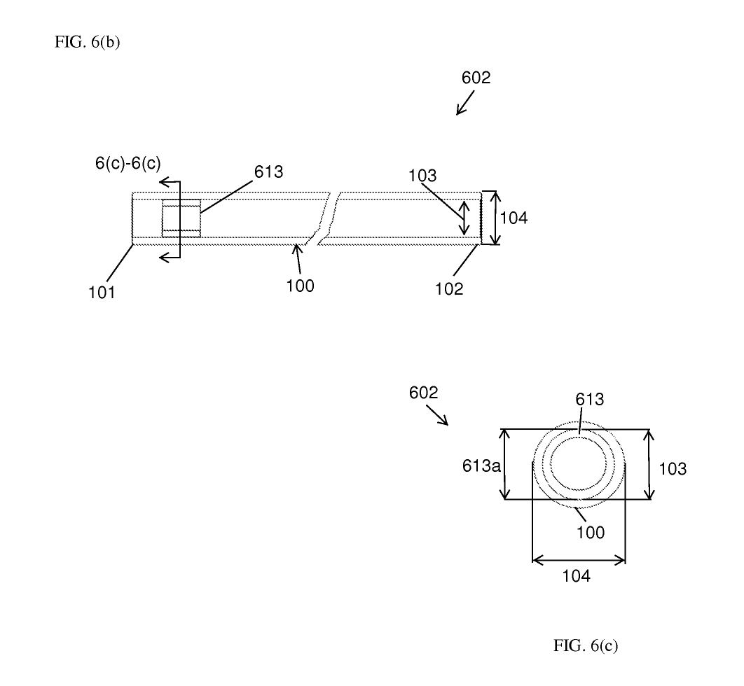

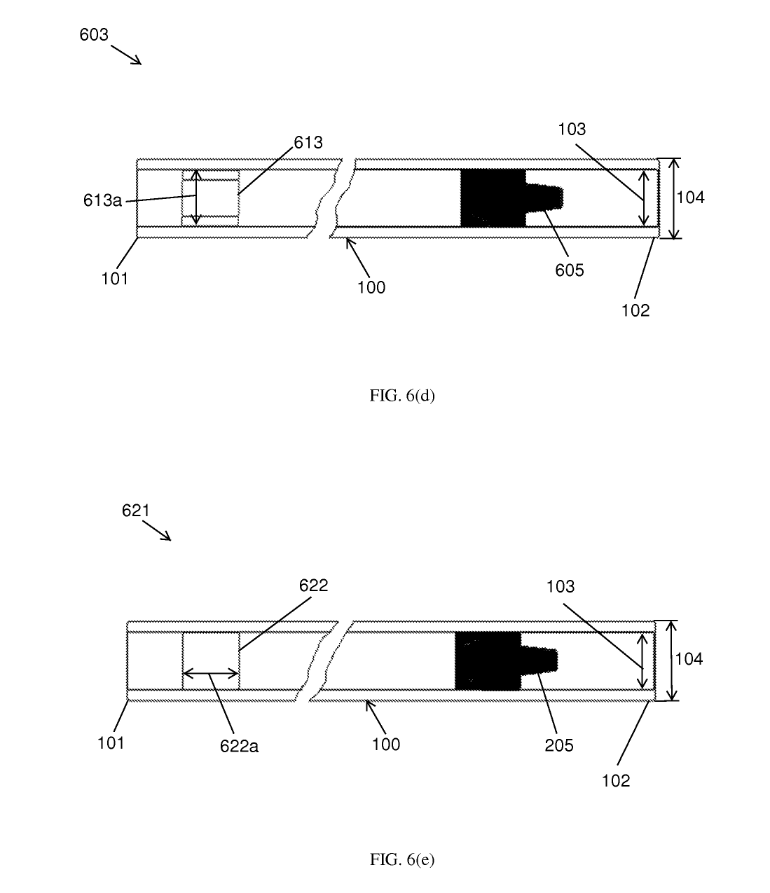

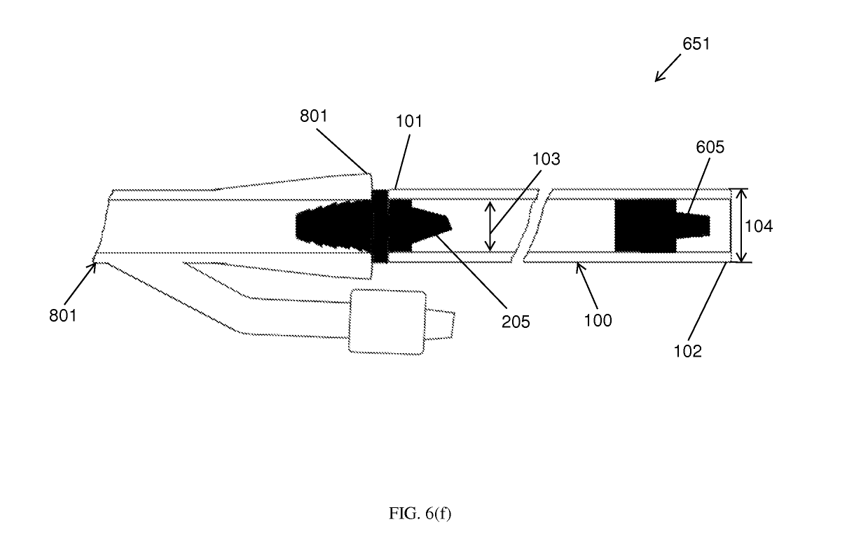

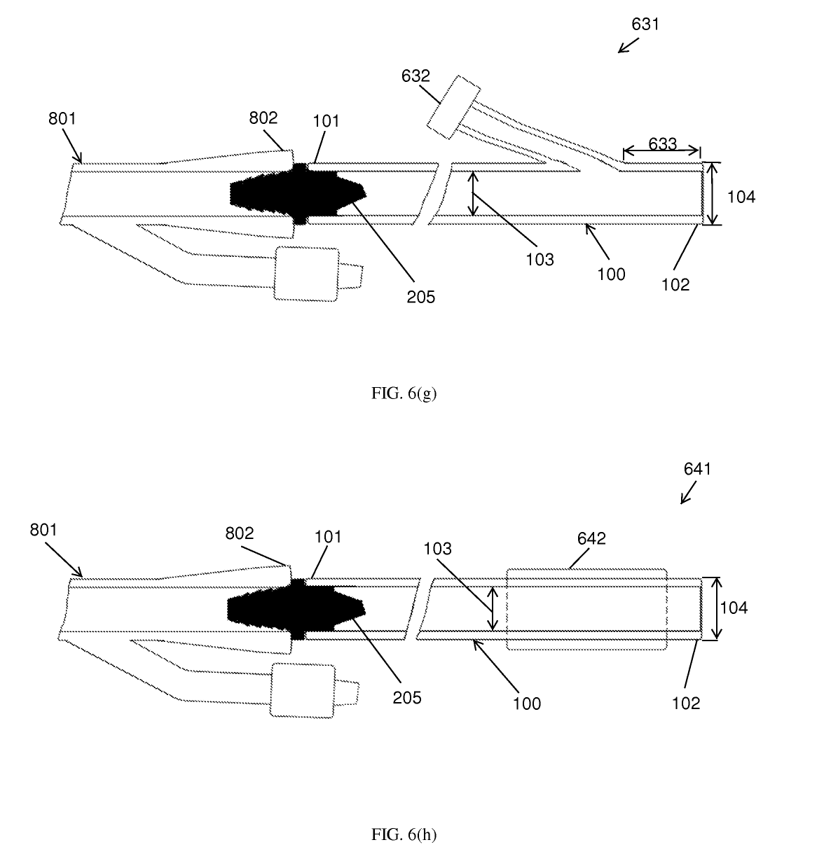

In various exemplary embodiments, there may be more than one connector as a component of tubing 100. Additional such connectors may or may not include a check-valve. In various exemplary embodiments, there may be other means for mitigating against microbial migration in a direction opposite of intended flow. For example, there may be more than one check-valve. The means for mitigating against microbial migration in the direction opposite of intended flow may be selected from one or more of the group comprising at least one check-valve (205, 308, and 605), at least one biofilm abater 613, and a region treated with an antimicrobial coating (e.g. such as region 622). The region of tubing 100 may comprise a surface which may be wetted by fluid flowing within tubing 100, such as urine flowing downstream. Such means for mitigating against microbial migration in the direction opposite of intended flow may be located in tubing 100, at first terminal end 101, or at second terminal end 102. See the FIG. 6(f) discussion below for an embodiment including at least two check-valves (205 and 605). Biofilm abaters 613 are discussed in the FIG. 6(b), FIG. 6(c), and FIG. 6(d) discussions. And see the FIG. 6(e) discussion below for an embodiment including wettable region treated with an antimicrobial coating.

As depicted in FIG. 2(b), exemplary embodiment 201 of tubing 100 may comprise the additional component of tape 206b. Connector integrated with check-valve 205 while connected to first terminal end 101 may comprise a length of tape 206b wrapped around the connection between the first terminal end 101 and the exit port 802 to prevent the connection from becoming disengaged which the minimizes ingress of contaminants, such as microbes, into tubing 100. Securing the connection with tape 206b facilitates maintaining a closed-system. In various exemplary embodiments, when tape 206b has been wrapped around the connection, tape 206b may circumscribe a portion of terminal end 101 and a portion of exit port 802.

In various exemplary embodiments, tape 206b may comprise a bonding means for tape 206b gripping outside surfaces of exit port 802 and first terminal end 101. For example, such a bonding means may be formed by at least one side of tape 206b having an adhesive property.

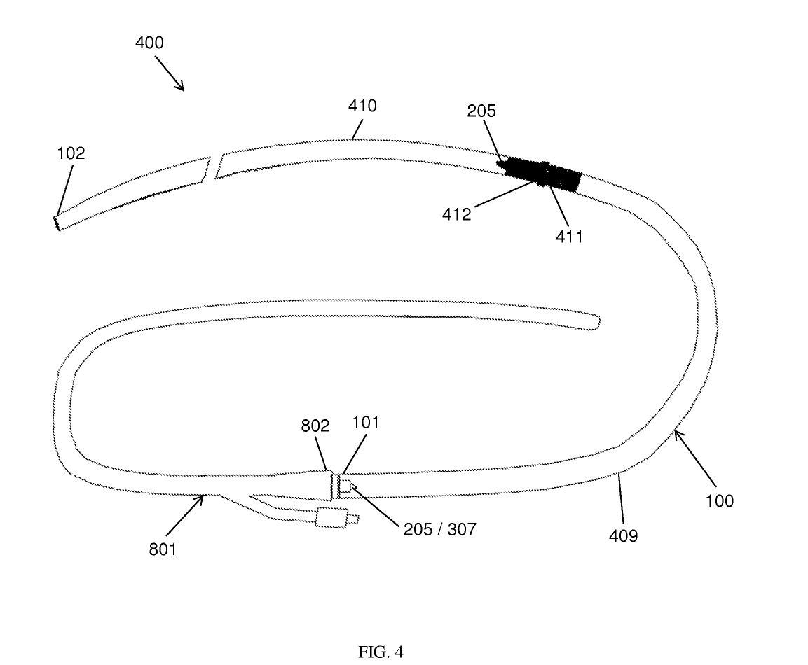

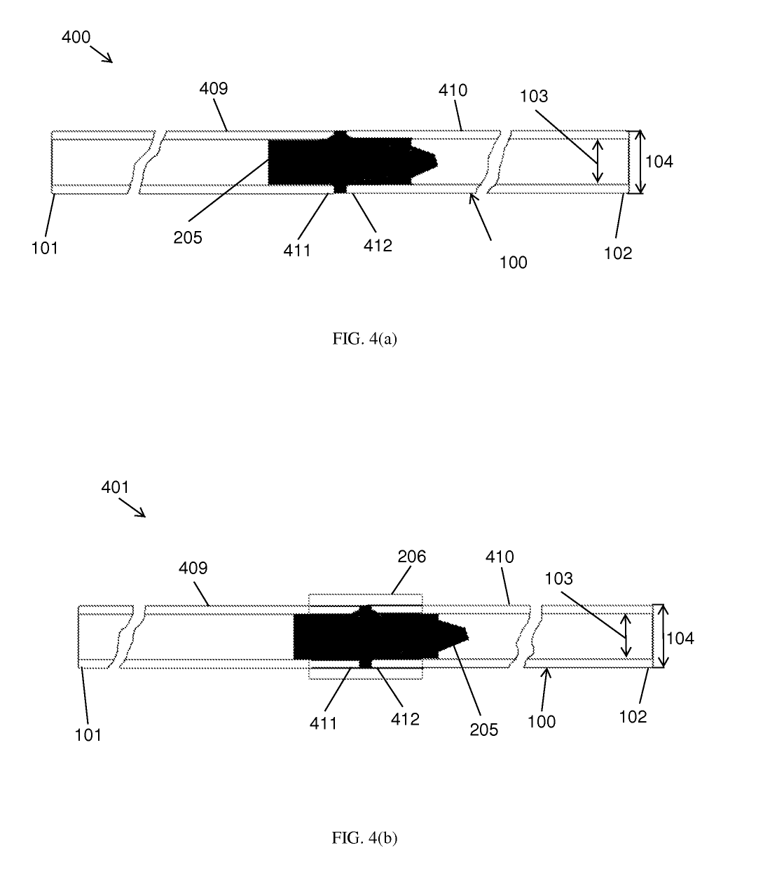

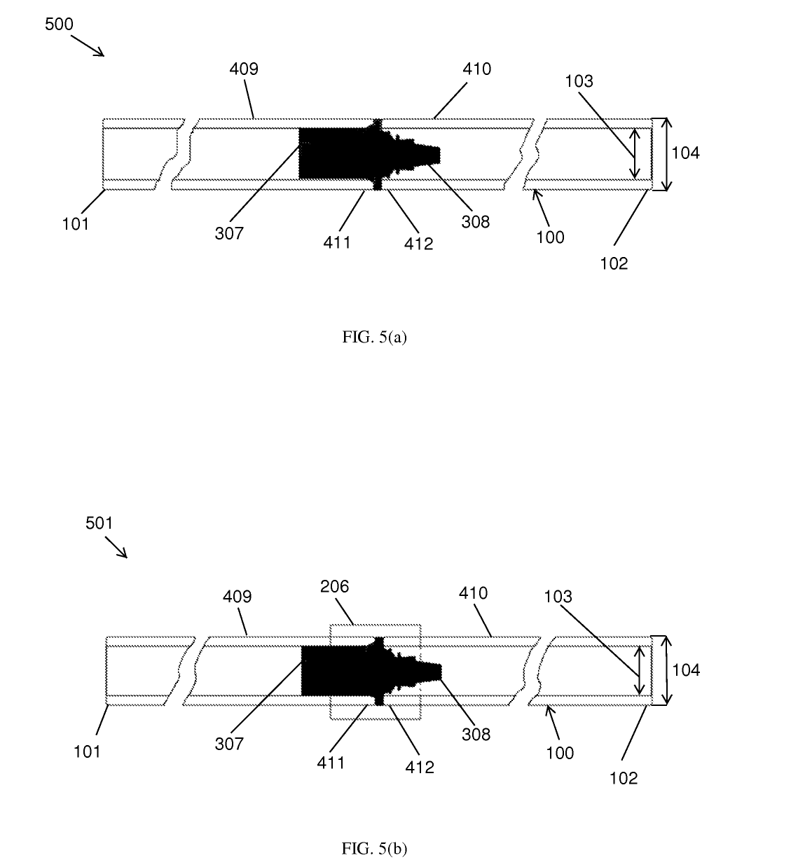

Tape 206b may be a sub-category of the broader category referred to as a “coupling sleeves” 206 within this specification. That is, coupling sleeve 206 may comprise tape 206b. See the discussion of the FIG. 4 and FIG. 5 series of figures for further details regarding coupling sleeve 206.

In various exemplary embodiments, tape 206b may comprise a color on a side facing a viewer, where the purpose of such a color may be to warn a medical practitioner that terminal end 101 should not be disengaged from exit port 802, while catheter 801 may be inserted into patient 910's urethra without some precaution taken to prevent opening of the closed-system. For example, and without limiting the scope of the present invention, such a color might be a bright orange, yellow, or red. Product literature and media (e.g. product inserts, white papers, website media, etc.) may include instructions as to the warning not to disengage such colored tape 206b. Tape 206b may serve at least two functions: (1) to prevent opening of an otherwise closed-system; and (2) to warn against breaching an otherwise closed-system.

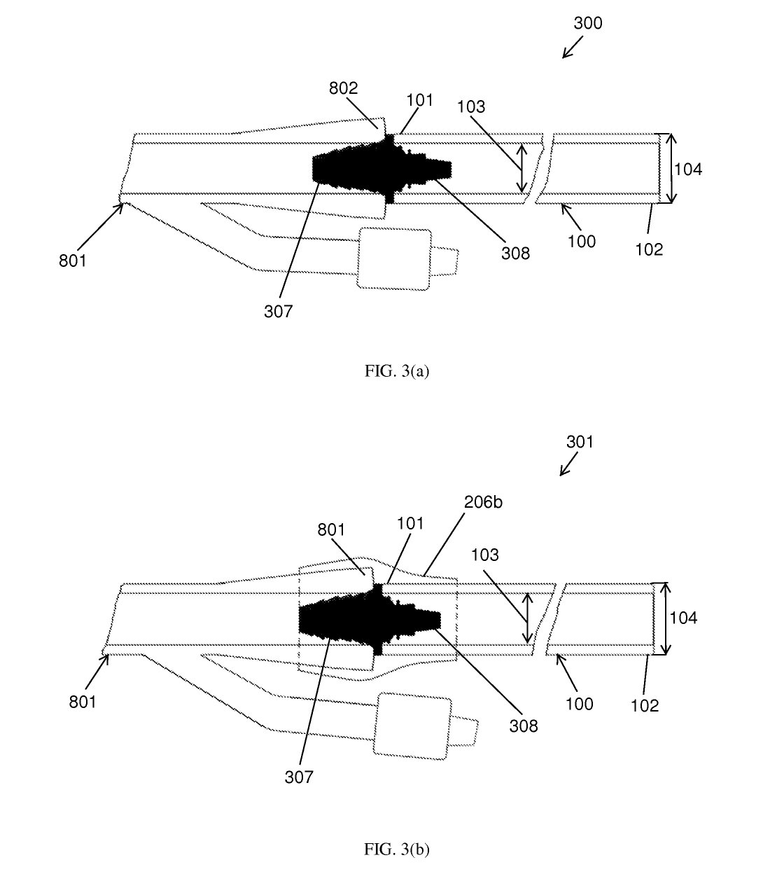

Now turning to the FIG. 3 series of figures. The FIG. 3 series of figures may be generally the same as the FIG. 2 series of figures, with the exception that connector integrated with check-valve 205 may be replaced with a connector 307 and a check-valve 308. FIG. 3(a) depicts exemplary embodiment 300 of tubing 100 which may comprise connector 307 coupled to non-integral check-valve 308, where first terminal end 101 of tubing 100 may be connected to exit port 802 of catheter 801 via connector 307, wherein connector 307 may be coupled to non-integral check-valve 308, shown from a longitudinal cross-sectional view.

In exemplary embodiment 300 (and 301), tubing 100 may comprise at least one connector 307 coupled to check-valve 308, where connector 307 and check-valve 308 may be separate articles of manufacture which may be coupled together in tubing 100. Connector 307 with coupled check-valve 308 may be connected to first terminal end 101 and may be configured to connect first terminal end 101 to exit port 802 of catheter 801.

In various exemplary embodiments, such a coupling of connector 307 to check-valve 308 may be permanent. Whereas, in other embodiments, such a coupling may be removable, i.e. after coupling, check-valve 308 may be disengaged from connector 307.

Various means may be used to couple connector 307 to non-integral check-valve 308. Connector 307 may frictionally hold check-valve 308. Check-valve 308 may be snapped into connector 307. Connector 307 may be solvent bonded to check-valve 308, when both components are constructed of appropriate polymers which may be solvent bonded together. Connector 307 may be ultrasonically welded to check-valve 308. Connector 307 may be glued to check-valve 308 using an appropriate adhesive (e.g. via a medical grade cyanoacrylate).

FIG. 3(b) depicts a similar embodiment shown in FIG. 2(b), with the inclusion of tape 206b wrapping around the connection. FIG. 3(b) depicts exemplary embodiment 301 of tubing 100 with an addition of tape 206b wrapping the connection between catheter 801 and tubing 100, shown from a longitudinal cross-sectional view.

The FIG. 4 and FIG. 5 series of figures may introduce exemplary tubing embodiments (400, 401, 500, and 501) where a single length of tubing 100 may be cut into two smaller pieces of tubing and then joined back together via a connector with a check-valve to form a single length of tubing 100. Such exemplary embodiments (400, 401, 500, and 501) may then provide for the means for mitigating against microbial migration in a direction opposite of intended flow to be located within tubing 100; as opposed to locating the means for mitigating against microbial migration in a direction opposite of intended flow at either first terminal end 101 (e.g. as shown in the FIG. 2 and FIG. 3 series of figures) and/or at second terminal end 102.

Such exemplary embodiments (400, 401, 500, and 501) may not be mutually exclusive with locating the means for mitigating against microbial migration in a direction opposite of intended flow at either first terminal end 101 and/or second terminal end 102. Exemplary embodiments (400, 401, 500, and 501) may also comprise other means for mitigating against microbial migration in a direction opposite of intended flow which may be located at either first terminal end 101 and/or second terminal end 102.

FIG. 4 depicts exemplary embodiment 400 of tubing 100 where tubing 100 may be sub-divided at a joint into two smaller tubes (409 and 410) such that connector integrated with check-valve 205 may be inserted between the two smaller tubes (409 and 410) to join the two smaller tubes (409 and 410), which then form a complete length of tubing 100, shown from a top view.

In exemplary embodiment 400, tubing 100 may comprise: first tube 409, second tube 410, a joint, and connector integrated with check-valve 205. First tube 409 may comprise first terminal end 101 and a third terminal end 411. First terminal end 101 longitudinally opposes third terminal end 411. Second tube 410 may comprise second terminal end 102 and a fourth terminal end 412. Second terminal end 102 may longitudinally oppose fourth terminal end 412. As noted above, first terminal end 101 may longitudinally oppose second terminal end 102. The joint may be made between first tube 409 and second tube 410 to form tubing 100 by using connector integrated with check-valve 205 to connect third terminal end 411 to fourth terminal end 412.

As used in this specification, “joint” refers to joining first tube 409 to second tube 410; while “connection” may refer to connecting tubing 100 to catheter 801 or to connecting tubing 100 to urine bag 901.

In terms of tubing 400, the single length may be short to accommodate use with leg bag 901a. For example, and without limiting the scope of the present invention, single length of tubing 100 may be from two inches to seven inches. In other embodiments, the single length may be twenty-four inches or longer to accommodate use with bed bag 901b.

FIG. 4(a) depicts exemplary embodiment 400 of tubing 100, shown from a longitudinal cross-sectional view focusing on the joint between first tube 409 and second tube 410 that may be formed by connector integrated with check-valve 205 connecting third terminal end 411 to fourth terminal end 412.

FIG. 4(b) depicts exemplary embodiment 401 of tubing 100 where the joint may be circumscribed by coupling sleeve 206, shown from a longitudinal cross-sectional view. Tubing 100 may comprise coupling sleeve 206 which may circumscribe the joint between third terminal end 411 and fourth terminal end 412. Coupling sleeve 206 may be configured to grip the joint, such that coupling sleeve 206 translation along tubing 100 may be minimized, i.e. coupling sleeve 206 may not freely slide along the longitude of tubing 100. In some embodiments, such gripping may be accomplished by coupling sleeve 206 comprising geometry to frictionally grip tubing 100. For example, coupling sleeve 206 may comprise an inside diameter which may be sized to be substantially the same as outside diameter 104 of tubing 100, such that there may be friction between coupling sleeve 206 and tubing 100 when tubing 100 may be inserted into the inside diameter of coupling sleeve 206. In other embodiments, coupling sleeve 206 may grip the joint by the bonding means. The bonding means may be selected from one or more of the group comprising heat welding, ultrasonic welding, solvent bonding, chemical adhesives (including adhesive tape), and the like. Coupling sleeve 206 may be bonded to outside diameter 104 of tubing 100 in a region proximal to each side of the joint to prevent the joint from becoming disengaged which minimizes ingress of contaminants, such as microbes, into tubing 100.

Coupling sleeve 206 may comprise a length of tape 206b wrapped around the joint to prevent the joint from becoming disengaged which minimizes ingress of contaminants, such as microbes, into the tubing 100. Tape 206b may comprise a color, e.g. a bright color as in red, to serve as a warning to a viewer, such as a medical practitioner, that the joint should not be opened and disengaged.

Now turning to the FIG. 5 series of figures. The FIG. 5 series of figures may be generally the same as the FIG. 4 series of figures, with the exception that connector integrated with check-valve 205 may be replaced with connector 307 and check-valve 308.

FIG. 5(a) depicts exemplary embodiment 500 of tubing 100 where tubing 100 may be sub-divided at the joint into two smaller tubes (409 and 410) such that connector 307 with non-integral check-valve 308 may be inserted between the two smaller tubes (409 and 410) to join the two smaller tubes (409 and 410), which then may form tubing 100, shown from a longitudinal cross-sectional view.

The details regarding the coupling of connector 307 to check-valve 308 were first discussed above under the FIG. 3(a) discussion and that discussion may apply here for exemplary embodiment 500 depicted in FIG. 5(a) and of embodiment 501 depicted in FIG. 5(b).

FIG. 5(b) depicts exemplary embodiment 501 of tubing 100 of FIG. 5(a), where the joint may be circumscribed by coupling sleeve 206, shown from a longitudinal cross-sectional view. The details regarding coupling sleeve 206 were discussed above under the FIG. 4(b) discussion and that discussion may apply here for exemplary embodiment 501.

Now turning to the FIG. 6 series of tubing 100 exemplary embodiments. The FIG. 6 series of figures addresses at least seven distinct exemplary embodiments, which may be combined into various exemplary embodiments, also within the scope of the present invention.

FIG. 6(a) depicts exemplary embodiment 601 of tubing 100 where check-valve 605 may have been inserted into tubing 100 by pushing the check-valve into a desired location, shown from a longitudinal cross-sectional view.

In exemplary embodiment 601, tubing 100 may comprise check-valve 605. Check-valve 605 may be assembled into tubing 100 by pushing check-valve 605 inside tubing 100 to a desired location along a length of tubing 100 such that tubing 100 may frictionally grip check-valve 605 to maintain the desired location, while also forming a complete seal between a periphery of check-valve 605 (e.g. an outside diameter of check-valve 605) and inside of tubing 100 (e.g. an inside diameter 103 of tubing 100) where the check-valve 605 may be positioned. Check-valve 605 may comprise an outside diameter, as part of check-valve 605's periphery, which may be substantially similar to inside diameter 103 of tubing 100, such that check-valve 605 may not translate (slide) within tubing 100 unless a force may be applied to overcome the frictional gripping force. The nature of such frictional gripping may be to form a seal between the outside diameter of check-valve 605 with inside diameter 103 of tubing 100, such that fluid flowing through tubing 100 may not pass between the outside diameter of check-valve 605 and inside diameter 103 of tubing 100. Such a complete seal may also be formed with the aid of one or more o-rings (or gaskets) circumscribing outside diameter of check-valve 605.

In various exemplary embodiments, tubing 100 may first be heated to increase its pliability and to expand tubing 100, then subsequently check-valve 605 may be pushed inside tubing 100 to the desired location. Upon tubing 100 cooling, tubing 100 may contract and increase frictional gripping between tubing 100 and check-valve 605.

In some embodiments, check-valve 605 may also be positionally fixed within tubing 100 by ultrasonically welding, solvent bonding, and by use of chemical adhesives.

While only one check-valve 605 may be depicted in FIG. 6(a), such a method of positioning check-valves within tubing 100 may be used to place a plurality of check-valves (such as check-valve 605) within tubing 100.

Notes regarding check-valve 605 and check-valve 308: Check-valve 308 may refer to a check-valve that may be configured to couple with connector 307. Check-valve 605 may not necessarily include such a further limitation.

FIG. 6(b), FIG. 6(c), and FIG. 6(d) address exemplary embodiments where tubing 100 may comprise one or more biofilm abaters 613, which may reside within tubing 100.

FIG. 6(b) depicts exemplary embodiment 602 of tubing 100 with biofilm abater 613 inserted into tubing 100, shown from a longitudinal cross-sectional view. FIG. 6(c) depicts the exemplary embodiment of FIG. 6(b), but shown from a top cross-sectional view.

In exemplary embodiment 602, tubing 100 may comprise one or more biofilm abater 613. Each biofilm abater 613 may comprise a ring. The ring may have structure which comprises an inside diameter, outside diameter 613a, and a thickness which may be defined by the difference between outside diameter 613a and the inside diameter. Outside diameter 613a may be configured to fit within inside diameter 103 of tubing 100. Outside diameter 613a may be frictionally held in place in a desired location within inside diameter 103 of tubing 100. Such frictional gripping may be accomplished by outside diameter 613a being substantially similar, in terms of dimension, to inside diameter 103 of tubing 100. The ring of biofilm abater 613 may be in a desired conformation within tubing 100, such that a plane of outside diameter 613a may be perpendicular to a longitude of the tubing 100. The ring of biofilm abater 613 may comprise a longitude, wherein the longitude of the ring of biofilm abater 613 may be parallel to the longitude of tubing 100. The longitude of tubing 100 may include a length, and the longitude of the ring of biofilm abater 613 may also include a length, wherein the length of tubing 100 may be greater than the length of the ring of biofilm abater 613.

The ring of biofilm abater 613 may include surface areas covering the external surfaces of the ring. The ring of biofilm abater 613 may comprise an antimicrobial coating, covering surface areas of the ring. In various exemplary embodiments, the wettable surface areas of the ring of biofilm abater 613 may be coated with the antimicrobial coating. Outside diameter 613a may not be coated with the antimicrobial coating.

As noted above in the general discussion of biofilm abaters preceding the figures discussion, such an antimicrobial coating may prevent microbial biofilms from growing across the surface areas of the ring which have treated with such an antimicrobial coating by inhibiting microbial growth or by inhibiting microbial attachment. For example, and without limiting the scope of the present invention, such an antimicrobial coating may comprise silver (or a silver alloy) to inhibit growth. Antimicrobial properties may be achieved where the entire ring of biofilm abater 613 may be constructed of silver, a silver alloy, or another abating material. In other exemplary embodiments, such an antimicrobial coating may comprise a molecularly smooth chemical coating yielding a molecularly smooth surface which may reduce the ability of microbes to attached to the coated region.

FIG. 6(d) depicts exemplary embodiment 603 of tubing 100 with check-valve 605 and with biofilm abater 613 inserted into tubing 100 upstream of check-valve 605, shown from a longitudinal cross-sectional view. FIG. 6(d) in comparison to FIG. 6(b) and FIG. 6(c), includes an additional component of check-valve 605. The reason for such a spatial relationship may be as follows: an intended function of check-valve 605 may be to prevent urine backflow (reflux) which may then prevent microbes free floating and/or in suspension in urine from travelling towards patient 910 using tubing 100 as a conduit; however, such a check-valve may not prevent biofilm growth migration (movement) towards patient 901; and so biofilm abater 613 may be installed upstream of check-valve 605 to abate biofilm migration.

In other exemplary embodiments, such check-valves (as 205, 307, and 605) may comprise both the back-flow prevention function and biofilm migration prevent function by the check-valve having its wettable surfaces coated with the antimicrobial coating.

FIG. 6(e) depicts exemplary embodiment 621 of tubing 100 where inside surface region 622 of tubing 100 may comprise the antimicrobial coating, and where inside surface region 622 may be upstream of check-valve 605, shown from a longitudinal cross-sectional view. Exemplary embodiment 621 depicted in FIG. 6(e) may be similar to exemplary embodiment 603 depicted in FIG. 6(d), except here in FIG. 6(e) biofilm abater 613 may be replaced with inside surface region 622.

Inside surface region 622 may be a region of antimicrobial coating. Inside surface region 622 may be an example of the wetted region treated with the antimicrobial coating. As a wetted region treated with the antimicrobial coating, there may be a reduced likelihood of a microbial biofilm migrating across the wetted region treated with the antimicrobial coating. Inside surface region 622 may comprise geometry of circumscribing inside diameter 103 of tubing 100 for a sub-length 622a that may be less than a total length of the tubing 100. In some embodiments, sub-length 622a may be a substantially similar length as the total length of the tubing 100. Inside surface region 622 may be located upstream of check-valve 605.

In various exemplary embodiments, the wetted region treated with the antimicrobial coating may be selected from one or more of the group comprising at least one connector (e.g. 307), at least one check-valve (e.g. 205, 308, and/or 605), at least one biofilm abater 613, and/or inside surface region 622 of tubing 100. For example, and without limiting the scope of the present invention, any of the check-valves (205, 308, 605) depicted in the various figures may have been treated with the antimicrobial coating, particularly on the wettable surfaces. Likewise, any of the connectors (205 and 307) depicted in the various figures may have been treated with the antimicrobial coating, particularly on the wettable surfaces.

Note while more than one connector with or without check-valve, check-valve with or without connector, biofilm abater 613, and inside surface region 622 may be employed in various embodiments, there is a practical limitation to the number of such components which may be employed in any given embodiment. Such a numerical limitation arises in part because tubing 100 in any given application must have a finite total length, which is generally the length necessary to run from catheter 801 to urine bag 901, including some length for slack and ease of patient 910 movement. Such a numerical limitation may arise in the case of check-valves because each additional check-valve may increase the necessary fluid pressure to flow through all check-valves installed in serial fashion and the fluid pressure itself may a maximum pressure created by patient 910 urinating and/or by any static head of urine within catheter 801 and tubing 100. As the number of check-valves increases the greater the required pressure is needed to flow through a serial installment of check-valves.

FIG. 6(f) depicts exemplary embodiment 651 of tubing 100 where there are two check-valves (205 and 605) inserted into tubing 100, in serial fashion (i.e. one upstream and one downstream with respect to each other), shown from a longitudinal cross-sectional view. Connector integrated with check-valve 205 may be used to connect first terminal end 101 to exit port 802 of catheter 801. Check-valve 605 may be inserted and pushed into the desired location within tubing 100. In various exemplary embodiments, either one or both check-valves may also comprise the antimicrobial coating, particularly on wettable surfaces.

FIG. 6(g) depicts exemplary embodiment 631 of tubing 100 which may comprise sampling port 632, shown from a longitudinal cross-sectional view. In exemplary embodiment 631, tubing 100 may comprise sampling port 632. Sampling port 632 may be located a linear distance 633 from second terminal end 102. Sampling port 632 may be configured to receive a syringe for the purpose of a taking a sample of fluid from within tubing 100. For example, and without limiting the scope of the present invention, a medical practitioner might take a urine sample from sampling port 632 in order to determine the microbial load present within the urine or for various other purposes.

In various exemplary embodiments, sampling port 632 may be located closer to second terminal 102 than to first terminal end 101. Linear distance 633 may be 0.25 to 7.00 inches in some embodiments and other distances in other embodiments. Such a location may serve two purposes. First by placing sampling port 632 closer to second terminal end 102 (and farther from first terminal end 101), there may be less interference with patient 910's comfort when urine samples are withdrawn from tubing 100, as movement of tubing 100 at the second terminal end 102 may be less likely to be communicated up tubing 100 to catheter 801. Secondly, taking urine samples from tubing 100 may constitute a technical, albeit intermittent, breach of what may have otherwise been a closed-system. Withdrawing urine samples from tubing 100 may increase the likelihood of introducing unwanted contaminants, such as microbes, into tubing 100. Placing sampling port 632 farther away from catheter 801 and patient 910, there may be a greater likelihood of minimizing any such contaminant reaching catheter 801 or patient 910.

FIG. 6(h) depicts exemplary embodiment 641 of tubing 100 which may comprise a graphical indicator 642, which may indicate which end of tubing 100 may be detached from urine bag 901, shown from a longitudinal cross-sectional view. Second terminal end 102 may comprise graphical indicator 642 to indicate which end of tubing 100 may be removably coupled to urine bag 901. In various exemplary embodiments, graphical indicator 642 may be located a proximal distance from the connection of second terminal end 102 and urine bag 901, such as from 0.125 to 7.000 inches from this connection, i.e. graphical indicator 642 may be located relatively close to this connection. In other embodiments, different dimensions for the proximal distance may be employed. Second terminal end 102 may be defined by a region which encompasses this connection and graphical indicator 642. Graphical indicator 642 may be located closer to this connection than to first terminal end 101.

In various exemplary embodiments, graphical indicator 642 may comprise tape, such as an adhesive tape, which may be wrapped around tubing 100 in the vicinity of second terminal end 102. In such embodiments, the tape does not necessarily have to wrap the connection itself, as the intent of such tape may not be to prevent disengagement of the connection, but rather to indicate that such an end may be appropriately and safely disengaged and still maintain a closed-system.

In various exemplary embodiments, graphical indicator 642 may be a color whereby such a color choice may indicate to a viewer, such as a medical practitioner, that this end of tubing 100 may be opened and disengaged from urine bag 901, as long as proper steps are taken to minimize ingress of contaminants, such as microbes, into tubing 100. For example, and without limiting the scope of the present invention, such a color might be green. Such a color choice may also be explained in various product literature, such as product inserts and media which may be found online instructing proper use of tubing 100.

Having discussed and disclosed the inventive tubing in its various embodiments, this disclosure now turns to discussing the inventive systems which may comprise the inventive tubing as discussed above.

A system for forming and maintaining a closed-system with respect to tubing 100 connected to catheter 801 may comprise: tubing 100, a second connector (e.g. 307 or 205), and catheter 801. The entirety of componentry as depicted in FIG. 4 may depict such a system.

Tubing 100 may comprise: first terminal end 101, second terminal end 102, first tube 409, second tube 410, and a first connector with check-valve (e.g. 205 or 307 coupled with 308). First tube 409 may comprise first terminal end 101 and third terminal end 411. First terminal end 101 may longitudinally oppose third terminal end 411. Second tube 410 may comprise second terminal end 102 and fourth terminal end 412. Second terminal end 102 may longitudinally opposes fourth terminal end 412. The first connector may comprise a check-valve. Such a check-valve may either be connector integrated with check-valve 205 or connector 307 that has been coupled to check-valve 308. The first connector with check-valve may be used to connect third terminal end 411 to fourth terminal end 412, forming a joint between third terminal end 411 and fourth terminal end 412. First terminal end 101 may longitudinally oppose second terminal end 102.

In some embodiments, tubing 100 of the system may comprise coupling sleeve 206. Coupling sleeve 206 may circumscribe the joint. Coupling sleeve 206 may be configured to prevent third terminal end 411 from becoming disengaged from fourth terminal end 412. As noted above, coupling sleeve 206 may grip the joint (exterior of the joint) by the bonding means. Coupling sleeve 206 may comprise tape 206b. Tape 206b may be an adhesive tape. Tape 206b may be a bright color, such as red.

Catheter 801 may comprise exit port 802. A second connector may be used to connect first terminal end 101 to exit port 802 such that second terminal end 102 remains available to removably couple to urine bag 901.

The second connector may or may not comprise a check-valve. When the second connector has no check-valve, the second connector may be connector 307. When the second connector comprises a check-valve, the second connector may be connector integrated with check-valve 205 or the second connector may be connector 307 coupled to check-valve 308.

In various exemplary embodiments the various check-valves of the system may or may not comprise the antimicrobial coating. In various exemplary embodiments the system may comprise one or more biofilm abaters 613 located within tubing 100, and generally with at least one biofilm abater 613 located upstream of the first connector with check-valve. In various exemplary embodiments, the system may also comprise one or more inside surface region 622's, and generally with at least one inside surface region 622 located upstream of the first connector with check-valve. In various exemplary embodiments, the system may also comprise urine bag 901.

When urine bag 901 may be removed from second terminal end 102, the system may still be deemed closed from upstream of the first connector with check-valve that may be located within tubing 100; while open from downstream of the first connector with check-valve. In order to maintain the system closed from near second terminal end 102, when urine bag 901 may be removed, additional componentry (e.g. a clamp or a cap) and various exemplary methods may be employed to close second terminal end 102.

Having discussed and disclosed various exemplary inventive tubing embodiments and inventive systems, this disclosure now turns to various exemplary methods for forming and maintaining a closed-system with respect to tubing 100 connected to catheter 801.

A method for forming and maintaining a closed-system with respect to tubing 100 connected to catheter 801 may comprise the steps:

Step 1: Cutting a segment of urinary tubing 100 for a purpose of connecting the segment of urinary tubing 100 to catheter 801 and to a urine bag 901.

Step 2: Forming a first connection between the segment of urinary tubing 100 and catheter 801 by connecting first terminal end 101 of the segment of urinary tubing to exit port 802 of catheter 801 using a first connector. (Note, the first connector here in exemplary methods context is not the first connector discussed above in the exemplary systems discussion; rather the first connector here in the exemplary methods context is more akin to the second connector of exemplary systems discussion.)

Step 3: Wrapping the first connection with a first piece of tape 206b to prevent the first connection from becoming disengaged which minimizes ingress of contaminants, such as microbes, into the closed-system. This step may be optional, yet the step may be important.

Second terminal end 102 of the segment of urinary tubing 100 may be available for connection to urine bag 901. Second terminal end 102 of the segment of urinary tubing 100 may be removably connected to urine bag 901. Also with respect to Step 3, as discussed above, tape 206b may be colored, such as red, to indicate to a viewer to not disengage first terminal end 101 from exit port 802 unless it may be time for catheter removal or other steps are taken to maintain the system as closed.

In various exemplary embodiments there may an additional step which precedes Step 2, wherein before making the first connection, exit port 802, first terminal end 101, and the first connector are sterilized by treating each component with a sterilizing material. Such treating may be immersing the component within the sterilizing material. Or treating may be wiping the component down with the sterilizing material. The sterilizing material may comprise a liquid, foam, or towel wetted with the liquid or foam. The liquid or the foam may be various alcohols (e.g. isopropyl), bleach, peroxides, betadine, and the like.

The first connector may comprise a check-valve. The first connector may either comprise a check-valve such that the first connector and the check-valve are integral being a single article of manufacture, i.e. first connector may be connector integrated with check-valve 205. Or, the first connector may comprise a non-integral check-valve which is coupled to the first connector, i.e. first connector may be connector 307 coupled to check-valve 308.

The method for forming and maintaining a closed-system with respect to tubing 100 connected to catheter 801 may comprise the following additional steps:

Step A: Cutting the segment of urinary tubing 100 into first tube 409 and second tube 410. First tube 409 may comprise first terminal end 101 and third terminal end 411. First terminal end 101 may longitudinally oppose third terminal end 411. Second tube 410 may comprise second terminal end 102 and fourth terminal end 412. Second terminal end 102 may longitudinally oppose fourth terminal end 412.

Step B: Forming a second connection between first tube 409 and second tube 410 by using a second connector to connect third terminal end 411 to fourth terminal end 412. (Note, the second connector here in exemplary methods context is not the second connector discussed above in the exemplary systems discussion; rather the second connector here in the exemplary methods context is more akin to the first connector of exemplary systems discussion.)

In various exemplary embodiments, Step A and Step B may proceed Step 2. In various exemplary embodiments, before making the second connection in Step B, third terminal end 411, fourth terminal end 412, and the second connector may be sterilized by treating each of the components with the sterilizing material.

In various exemplary embodiments, the second connection may comprise the step of securing the second connection with coupling sleeve 206. Coupling sleeve 206 may circumscribe the joint between third terminal end 411 and fourth terminal end 412. Coupling sleeve 206 may be configured to prevent third terminal end 411 from becoming disengaged from fourth terminal end 412 which may minimize ingress of contaminants, such as microbes, into the closed-system.

The step of securing coupling sleeve 206 to the joint may involve wrapping tape 206b around the joint, i.e. coupling sleeve 206 may comprise tape 206b, which may be an adhesive tape. Tape 206b may be colored, such as red, to indicate to a viewer that the second connection should not be opened unless intended to open the system.

Alternatively, the step of securing coupling sleeve 206 to the joint may involve the step of bonding coupling sleeve 206 to outside diameter 104 of tubing 100 in a region proximal to each side of the joint to prevent the second connection from becoming disengaged. Such bonding may be accomplished by the bonding means, e.g. of ultrasonic welding, solvent bonding, use of chemical adhesives, and the like.

The second connector may comprise a check-valve. The second connector may either comprise a check-valve such that the second connector and the check-valve are integral being a single article of manufacture, i.e. second connector may be connector integrated with check-valve 205. Or, the second connector may comprise a non-integral check-valve which may be coupled to the second connector, i.e. the second connector may be connector 307 coupled to check-valve 308.

The method for forming and maintaining a closed-system with respect to tubing 100 connected to catheter 801 may comprise the following additional steps:

Step 4: Attach graphical indicator 642 to outside diameter of tubing 104 at the proximal distance from second terminal end 102. See FIG. 6(h) and the above discussion of graphical indicator 642. In some embodiments, graphical indicator 642 may be green colored adhesive tape. In some embodiments, Step 4 may be optional.

Step 5: When urine bag 901 may be changed by removing urine bag 901 and replacing urine bag 901 with a new urine bag, the segment of urinary tubing 100 may be clamped shut with a clamp prior to removal of urine bag 901. The clamp may remain in place until the new urine bag may be attached to second terminal end 102 at which point the clamp may be removed. It may be desirable to clamp shut tubing 100 as near as possible to second terminal end 102 without interfering with the mechanics of removing and attaching urine bag 901. In some embodiments, Step 5 may be optional.

In various exemplary methods, the clamping step may be replaced with a capping step. When urine bag 901 may be changed by removing urine bag 901 and replacing urine bag 901 with a new urine bag, second terminal end 102 may be capped shut with a cap, preferably a sterile cap, prior to removal of urine bag 901. The cap may remain in place until the new urine bag may be attached to second terminal end 102 at which point the cap may be removed.

A tubing for mitigating against microbial migration, as well as a system and method for forming and maintaining a closed-system of urinary tubing have been described. The foregoing description of the various exemplary embodiments of the invention has been presented for the purposes of illustration and disclosure. It is not intended to be exhaustive or to limit the invention to the precise form disclosed. Many modifications and variations are possible in light of the above teaching without departing from the spirit of the invention.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

What is claimed is:

1. A tubing configured to mitigate against microbial migration in a direction opposite of intended flow, wherein the tubing comprises:
a first terminal end;
a second terminal end, longitudinally opposing the first terminal end;
a wettable region, wherein the wettable region comprises a surface which is wetted by a fluid flowing within the tubing; and
a means for mitigating against microbial migration in a direction opposite of intended flow;
wherein the means for mitigating against microbial migration in the direction opposite of intended flow is selected from one or more of the group consisting of at least one check-valve, at least one biofilm abater, and the wettable region treated with an antimicrobial coating; and
wherein the means for mitigating against microbial migration in the direction opposite of intended flow are located in the tubing, at the first terminal end, or at the second terminal end.

2. The tubing according to claim 1, wherein one or more of the tubing, the at least one check-valve, and the at least one biofilm abater are substantially constructed of a polymer; wherein the polymer is urethane, rubber, polyvinyl chloride, silicone, polyethylene, nylon, fluropolymers, acrylic or polycarbonate.

3. The tubing according to claim 2, wherein the polymer comprises antimicrobial properties.

4. The tubing according to claim 1, wherein the wetted region treated with the antimicrobial coating is selected from one or more of the group consisting of at least one connector, the at least one check-valve, the at least one biofilm abater and an inside surface region of the tubing; wherein the inside surface region circumscribes an inside diameter of the tubing for a sub-length of the tubing; wherein the wetted region treated with the antimicrobial coating reduces the likelihood of a microbial biofilm migrating across the wetted region treated with the antimicrobial coating.

5. The tubing according to claim 4, wherein the tubing further comprises the at least one check-valve; wherein the inside surface region is located upstream of the at least one check-valve.

6. The tubing according to claim 1, wherein the tubing further comprises the at least one check-valve; wherein the at least one check-valve is a separate component assembled into the tubing by pushing the at least one check-valve inside the tubing to a desired location along a length of the tubing such that the tubing frictionally grips the check-valve to maintain the desired location, while also forming a complete seal between a periphery of the at least one check-valve and inside of the tubing where the at least one check-valve is positioned.

7. The tubing according to claim 1, wherein the tubing further comprises at least one connector integrated with a check-valve selected from the at least one check-valve, such that the at least one connector integrated with the check-valve are a single article of manufacture; wherein at least one of the at least one connector integrated with the check-valve is connected to the first terminal end and is configured to connect the first terminal end to an exit port of a catheter.

8. The tubing according to claim 1, wherein the tubing further comprises at least one connector coupled to a check-valve selected from the at least one check-valve wherein the at least one connector and the check-valve are separate articles of manufacture which are then coupled together; wherein one of the at least one connector with coupled check-valve is connected to the first terminal end and is configured to connect the first terminal end to an exit port of a catheter.

9. The tubing according to claim 1, wherein the tubing further comprises:
a first tube with the first terminal end and a third terminal end, such that the first terminal end and third terminal end longitudinally oppose each other; and
a second tube with a fourth terminal end and the second terminal end, such that the fourth terminal end and the second terminal end longitudinally oppose each other; and
wherein a joint is made between the first tube and the second tube to form the tubing by using a connector to connect the third terminal end to the fourth terminal end.

10. The tubing according to claim 9, wherein the tubing further comprises a coupling sleeve, wherein the coupling sleeve circumscribes the joint between the third terminal end and the fourth terminal end; and wherein the coupling sleeve is configured to grip the joint by a bonding means such that the coupling sleeve translating along the tubing is minimized.

11. The tubing according to claim 9, wherein the connector further comprises a check-valve selected from the at least one check-valves such that the connector and the check-valve are integral with respect to each other being a single article of manufacture.

12. The tubing according to claim 9, wherein the connector further comprises a check-valve selected from the at least one check-valves, wherein the check-valve is a separate component which is coupled the connector.

13. The tubing according to claim 1, wherein the at least one biofilm abater further comprises:
a ring, wherein the ring further comprises:
an outside diameter, such that the outside diameter is configured to fit within an inside diameter of the tubing and be frictionally held in place in a desired location within the inside diameter and in a desired conformation such that a plane of the outside diameter is perpendicular to a longitude of the tubing;
an antimicrobial coating which covers surface areas of the ring;
wherein the antimicrobial coating prevents microbial biofilms from growing across the surface areas of the ring.

14. The tubing according to claim 1, wherein the tubing further comprises a sampling port, wherein the sampling port is a linear distance from the second terminal end, wherein the sampling port is closer to the second terminal end than to the first terminal end; and wherein the sampling port is configured to receive a syringe for the purpose of taking a sample of fluid from within the tubing.

15. A method for forming and maintaining a closed-system with respect to urinary tubing connected to a catheter comprising the steps:
cut a segment of urinary tubing for a purpose of connecting the segment of urinary tubing to the catheter and to a urine bag, wherein the segment of urinary tubing comprises a first terminal end disposed opposite of a second terminal end; and
before making any connections, treat the following components with a sterilizing material: an exit port of the catheter, the first terminal end of the segment of urinary tubing, and a first connector; and
form a first connection between the segment of urinary tubing and the catheter by connecting the first terminal end of the segment of urinary tubing to the exit port of the catheter using the first connector; and
wrap the first connection with a first piece of tape to prevent the first connection from becoming disengaged which minimizes ingress of contaminants into the closed-system; and
wherein a second terminal end of the segment of urinary tubing is removably connected to a urine bag.

16. The method according to claim 15, wherein when the urine bag is changed by removing the urine bag from the second terminal end and replacing the urine bag with a new urine bag, the segment of urinary tubing is clamped shut with a clamp prior to removal of the urine bag; wherein the clamp remains in place until the new urine bag is attached to the second terminal end at which point the clamp is removed.

17. The method according to claim 15, wherein the first connector further comprises a check-valve such that the first connector and the check-valve are integral being a single article of manufacture.

18. The method according to claim 15, wherein the first connector further comprises a check-valve which is coupled to the first connector.

19. The method according to claim 15, wherein the segment of urinary tubing is further prepared to form and maintain a closed-system, comprising the steps:
cutting the segment of urinary tubing into a first tube and a second tube, wherein the first tube comprises the first terminal end and a third terminal end disposed opposite of the first terminal end; wherein the second tube comprises the second terminal end disposed opposite of a fourth terminal end; and
before making a second connection, treat the following components with the sterilizing material: a second connector, the third terminal end and the fourth terminal end; and
form the second connection between the first tube and the second tube by using the second connector to connect the third terminal end to the fourth terminal end.

20. The method according to claim 19, wherein the method further comprises securing a coupling sleeve to the second connection, such that the coupling sleeve circumscribes a joint between the third terminal end and the fourth terminal end, wherein the coupling sleeve is configured to prevent the third terminal end from becoming disengaged from the fourth terminal end which minimizes ingress of contaminants into the closed-system by utilizing a bonding means to secure the coupling sleeve to the third terminal end and to the fourth terminal end.

21. The method according to claim 19, wherein the method further comprises securing a graphical indicator within a proximal distance of the second terminal end to indicate which end of the tubing is removably coupled to a urine bag.

22. The method according to claim 19, wherein the second connector further comprises a check-valve such that the second connector and the check-valve are integral being a single article of manufacture.

23. The method according to claim 19, wherein the second connector further comprises a check-valve which is coupled to the second connector.

24. A system for forming and maintaining a closed-system with respect to urinary tubing connected to a catheter comprising:
a segment of urinary tubing, which further comprises:
a first tube comprising a first terminal end and a third terminal end, wherein the first terminal end longitudinally opposes the third terminal end;
a second tube comprising a second terminal end and a fourth terminal end, wherein the second terminal end longitudinally opposes the fourth terminal end, and wherein the first terminal end longitudinally opposes the second terminal end;
a first connector further comprising a check-valve; and
a coupling sleeve;
a catheter, wherein the catheter further comprises an exit port; and
a second connector;
wherein the first connector connects the third terminal end to the fourth terminal end forming a joint between the third terminal end and the fourth terminal end;
wherein the coupling sleeve circumscribes the joint, wherein the coupling sleeve is configured to prevent the third terminal end from becoming disengaged from the fourth terminal end;
wherein the second connector is used to connect the first terminal end to the exit port; and
wherein the second terminal end is removably coupled to a urine bag.
